(19) United States
(12) Patent Application Publication
(10) Pub. No.: US 2020/0251192 A1
(43) Pub. Date: Aug. 6, 2020

(54) MEDICAL DISPLAY SYSTEM AND METHOD

(71) Applicant: Canon Medical Systems Corporation, Otawara-shi (JP)

(72) Inventor: Steven REYNOLDS, Edinburgh (GB)

(73) Assignee: Canon Medical Systems Corporation, Otawara-shi (JP)

(21) Appl. No.: 16/262,986

(22) Filed: Jan. 31, 2019

(51) Int. Cl. G16H 10/40; G16H 10/60; G16H 50/20

(52) U.S. Cl. CPC G16H 10/40; G16H 50/70; G16H 50/20; G16H 10/60

(57) ABSTRACT

A medical information system comprises acquisition circuitry configured to acquire information concerning a subject from at least one memory, the information concerning the subject comprising a plurality of data points each having an associated time value; display circuitry configured to display the information concerning the subject along a time axis; and processing circuitry configured to: classify the information concerning the subject; and determine a granularity of clustering of the data points on the time axis in dependence on the classification of the information concerning the subject, such that data points in different regions of the time axis are clustered with different granularity.

### FIELD

Embodiments described herein relate generally to a system and method for the display of medical data, for example a system and method for displaying medical information along a non-linear timeline.

### BACKGROUND

The amount of data captured for a given patient by hospital systems is ever increasing. As the amount of data increases, it may become more and more challenging to present this data in an optimal way to communicate the right information at the right time to make clinical decisions.

In some known applications, a display screen showing a timeline is used to present events to a user, for example a clinician. The timeline may also be referred to as a time axis.

The events may comprise, for example, medical interventions, lab results, patient data measured, and imaging data acquisitions. Each event may be represented by a respective icon or marker which is positioned relative to the timeline.

The timeline is scaled and is marked in regular intervals such that each interval along the timeline represents a given amount of time. For example, a timeline may show time in hours, days or months. The interval of time that is shown on the display screen may be referred to as a time window.

The user may navigate through the events by panning along the timeline and by zooming the timeline. Panning changes the time window and allows a different part of the timeline to be shown. Zooming allow the timeline to be shown at a different scale. For example, a user may zoom in from a timeline in which time is shown in days to a timeline in which time is shown in hours.

It is known to cluster events that occurred at similar time. For example, events that occurred on the same day may be clustered together and may be represented on the timeline by a single icon or marker.

Clustering may be performed by counting events that fall into a given period of time, which may be referred to as a bucket. An icon or marker representing a cluster may include a count of the events included in the cluster.

In systems as described above, events outside the current time window are not displayed to the user. If the user wants to explore a patient's history at times outside the current time window, user interaction is required.

If the user zooms out to make historical data visible, precision and context on events at a given time point of interest may be lost or reduced.

### BRIEF DESCRIPTION OF THE DRAWINGS

Embodiments are now described, by way of non-limiting example, and are illustrated in the following figures, in which:

FIG. 1 is a schematic illustration of an apparatus in accordance with an embodiment;

FIG. 2 is a flow chart illustrating in overview a process performed in accordance with an embodiment;

FIG. 3 is an example of a non-linear timeline display in accordance with an embodiment;

FIG. 4a is a schematic representation of the projection of events onto a linear timeline;

FIG. 4b is a schematic representation of the projection of events onto a non-linear timeline;

FIG. 5 is a schematic representation of the projection of events onto a non-linear timeline having two focal points;

FIG. 6 is an example of a non-linear timeline display in accordance with an embodiment;

FIG. 7 is an example of a colored non-linear timeline display in accordance with an embodiment

FIG. 8a is a schematic representation of the coloring of a non-linear timeline; and

FIG. 8b is a schematic representation of the coloring of a non-linear timeline having two focal points.

### DETAILED DESCRIPTION

Certain embodiments provide a medical information system comprising: acquisition circuitry configured to acquire information concerning a subject from at least one memory, the information concerning the subject comprising a plurality of data points each having an associated time value; display circuitry configured to display the information concerning the subject along a time axis; and processing circuitry configured to: classify the information concerning the subject; and determine a granularity of clustering of the data points on the time axis in dependence on the classification of the information concerning the subject, such that data points in different regions of the time axis are clustered with different granularity.

Certain embodiments provide a medical information display method comprising: acquiring information concerning a subject from at least one memory, the information concerning the subject comprising a plurality of data points each having an associated time value; displaying the information concerning the subject along a time axis; and classifying the information concerning the subject; and determining a granularity of clustering of the data points on the time axis in dependence on the classification of the information concerning the subject, such that data points in different regions of the time axis are clustered with different granularity.

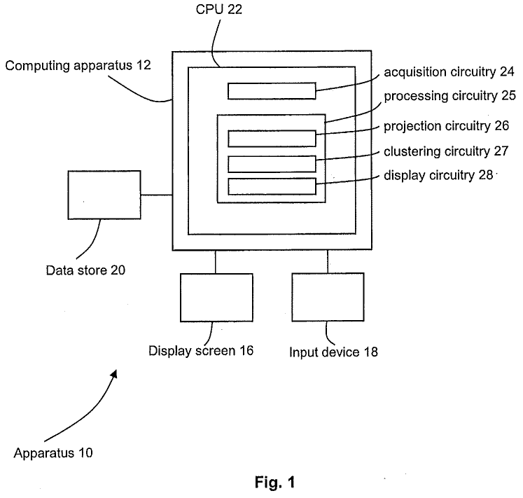

A medical information system 10 according to an embodiment is illustrated schematically in FIG. 1. In the present embodiment, the medical information system 10 is configured to present a display representing a plurality of medical events relating to a patient or other subject.

In other embodiments, the medical information system 10 is configured to present any appropriate medical data, where medical may include veterinary.

The medical information system 10 comprises a computing apparatus 12, which in this case is a personal computer (PC) or workstation. The computing apparatus 12 is connected to a display screen 16 or other display device, and an input device or devices 18, such as a computer keyboard and mouse. In alternative embodiments, the display screen 16 is a touch screen, which also acts as an input device 18. In some embodiments, the computing apparatus 12 is a mobile device, for example a smartphone or tablet computer. In some embodiments, the computing apparatus 12 comprises two or more computing devices, which may be connected by a cable or wirelessly.

The computing apparatus 12 receives medical data from a data store 20. In alternative embodiments, the medical information system 10 receives medical data from one or more further data stores (not shown) instead of or in addition to data store 20. For example, the medical information system 10 may receive medical data from one or more remote data stores (not shown) which may form part of a Picture Archiving and Communication System (PACS) or other information system, for example a laboratory data archive, an Electronic Medical Record (EMR) system, or an Admission Discharge and Transfer (ADT) system.

Computing apparatus 12 provides a processing resource for automatically or semi-automatically processing medical data. Computing apparatus 12 comprises a central processing unit (CPU) 22.

Computing apparatus 12 includes acquisition circuitry 24 configured to acquire medical information about a patient or other subject and processing circuitry 25 configured to process the medical information for display. The processing circuitry 25 comprises projection circuitry 26, clustering circuitry 27 and display circuitry 28.

In the present embodiment, the circuitries 24, 25, 26, 27, 28 are each implemented in computing apparatus 12 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 1 for clarity.

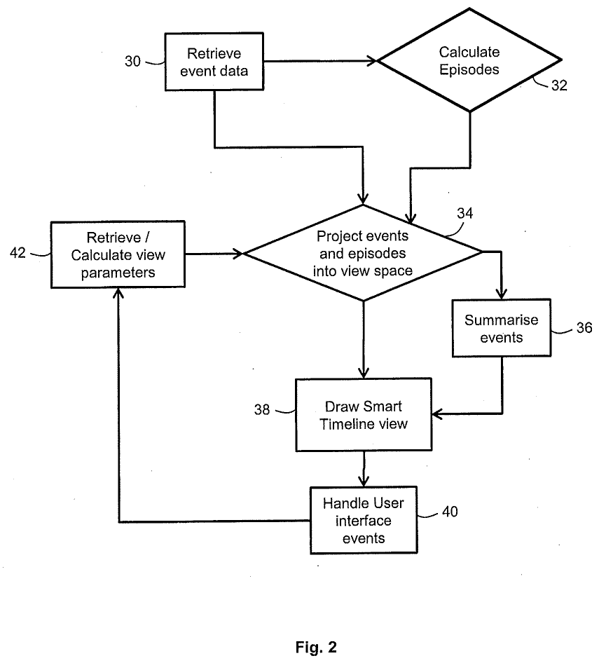

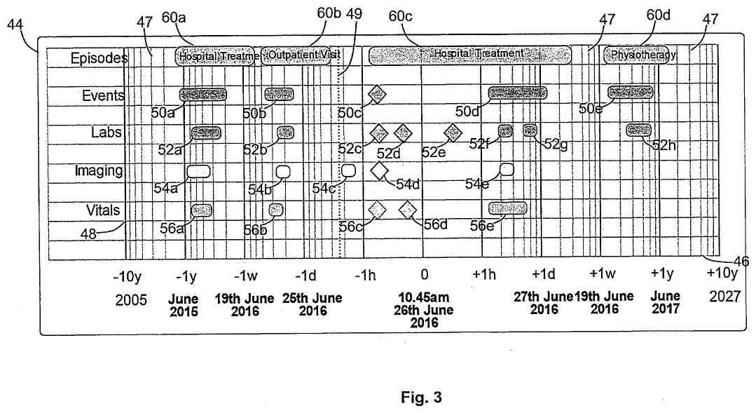

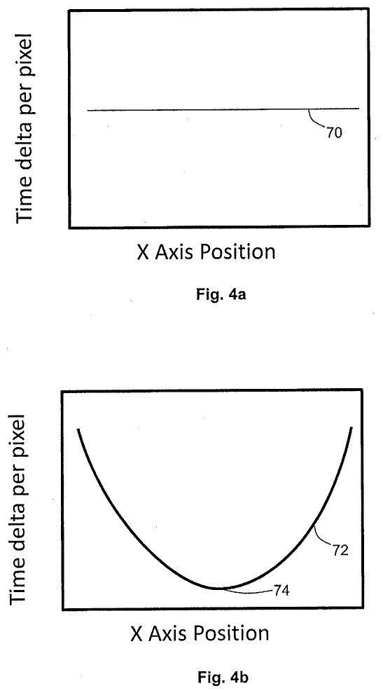

The system of FIG. 1 is configured to perform a series of stages as illustrated in overview in the flow chart of FIG. 2 with reference to the example display of FIG. 3. The series of stages of the flow chart of FIG. 2 include projecting medical events onto a non-linear timeline (which may also be referred to as a time axis). We consider the features of such a non-linear timeline with reference to FIGS. 3, 4a and 4b.

FIG. 3 shows a display 44 on which various medical episodes 60a to 60d and events 50a to 56e are plotted on a non-linear timeline 46. The display 44 may be shown on, for example, display screen 16.

In a conventional linear timeline (which may also be referred to as a time axis), the scale of the timeline is consistent along the timeline. On the timeline, intervals of equal length represent equal periods of time. The length of time represented by the width of each pixel of the timeline (which may be described as the time delta per pixel or time density) is constant across the entire length of the timeline. For example, each pixel may represent an hour or a day of time. FIG. 4a is a schematic plot of time delta per pixel against x axis position for a linear timeline. Since each pixel represents the same amount of time (constant scale), the line 70 showing time delta per pixel against x axis position is flat.

In a non-linear timeline, the amount of time represented by each pixel of the timeline is not constant across the length of the timeline. Instead, the scale of the timeline varies with position on the timeline. For example, at one point on the timeline a pixel may represent an hour, at another point on the timeline a pixel may represent a day, and at a further point on the timeline a pixel may represent a week.

FIG. 4b is a schematic plot of time delta per pixel against x axis position for an example of a non-linear timeline. Line 72 represents the relationship between time delta per pixel and x axis position. It may be seen that the value for time delta per pixel varies with x axis position. At the left and right ends of the line 72, there is a high time delta per pixel. At the lowest point 74 on the line 72, there is a low time delta per pixel.

Point 74 may be representative of a time that is of particular interest, for example today's date, or the date of an important medical event. By changing the time delta per pixel with position on the x axis, events close in time to a time of interest may be shown in greater detail and at a larger scale than events that occurred further from the time of interest.

In the example shown in FIG. 4b, the time delta per pixel varies smoothly with position on the x axis, so that the scale of the time axis is smaller the further away the position on the x axis is from the time of interest. In further embodiments, the time delta per pixel may be any suitable function of the x axis position along the timeline.

In some embodiments, at least one section of the time delta per pixel against x axis position curve may be flat, such that time delta per pixel is constant over at least one section of the timeline.

In some embodiments, the time of interest is an extended time period of interest rather than a single time point. In such embodiments, the scale may be kept constant throughout the time period of interest (causing the plot of time delta per pixel against x axis position to have a flat region), and may then decrease in proportion to distance from the time period of interest. In such embodiments, the plot of time delta per pixel versus x axis position may include a linear region at the time period of interest.

In some embodiments time delta per pixel reaches a maximum value at a high and/or low value of x axis position and is then kept constant at the maximum value.

In some embodiments, the time delta per pixel may change in a stepwise fashion rather than smoothly. In some embodiments, the function of time delta per pixel versus x axis position is discontinuous.

Although scale is described in pixels in the above description, scale may also be described in terms of any units of length and/or position on the timeline. Although timelines are shown in the figures as horizontal, they may also be vertical or in any suitable orientation.

We turn to the time axis 46 shown in the example of FIG. 3. The time axis 46 represents a portion of a patient's lifetime. The time axis includes both past and future dates.

In the example of FIG. 3, the time point of interest is 10.45 am on the 26 Jun. 2016. The time point of interest is positioned in the center of the time axis. The time axis is labelled with absolute times (for example, 2005, June 2015, 19 Jun. 2016, 25 Jun. 2016), The time axis is also labelled with times relative to the time point of interest (for example, −10 years, −1 year, −1 week, −1 day, −1 hour).

The origin 0 of the time axis is at the time point of interest. The time point of interest may also be referred to as the focal point.

A granularity of the time axis varies with distance from the focal point. At or near the focal point, events can be seen in greatest detail, so the granularity of the time axis is high. Further from the focal point, events are shown in less detail, so the granularity of the time axis is low.

A time marker 49 is represented in FIG. 3 as a dotted line. The time marker 49 may be used to mark a selected time on the timeline. For example, the time marker 49 may mark a time that corresponds to a current mouse position.

In the plot 44 of FIG. 3, the vertical axis 48 is representative of a plurality of different categories, each of which is represented by a respective horizontal band of the plot 44.

The first category shown on vertical axis 48 is Episodes. Episodes may represent any appropriate time periods, for example any time periods that are represented in the patient's medical record.

An episode may correspond to a period of treatment. For example, an episode may correspond to a period of inpatient treatment or outpatient treatment. An episode may correspond to a period of delivery of a particular medication, or of a treatment such as physiotherapy.

Episodes are represented in a first horizontal band of the plot 44. Each episode is representative of a respective period of time. The period of time is represented by an extended marker (in this example, a rounded rectangular marker) which extends from a start time to a finish time, shown relative to the time axis. An episode may relate to any appropriate time period in a patient's (or other subject's) medical history.

In some circumstances, episodes may be used to provide an overview of medical interactions with the patient, for example an indication of when the patient has been in hospital and when the patient has been out of hospital.

In the example shown in FIG. 3, a first episode 60a is representative of hospital treatment. The first episode extends from just before the −1 year mark to just after the −1 week mark, and so represents a lengthy stay in hospital.

A second episode 60b is representative of outpatient treatment. The second episode 60b extends from 4 days ago to a few hours ago.

A third episode 60c is representative of a further period of hospital treatment, which extends across the time point of interest. A third episode 60d is representative of a planned period of physiotherapy.

It may be seen that episodes having similar length on the display may represent very different lengths of time, depending on where the episodes are positioned on the non-linear timeline. Episodes are displayed upon the timeline at a scale relevant to the position on the non-linear timeline.

The presence of episodes on the plot 44 may allow the user to put other events on the plot 44 in context, for example to ascertain whether events occurred during a period of hospital treatment or during a period of outpatient treatment.

In the example of FIG. 3, the plot 44 also includes four categories of events. Events relate to interactions with a healthcare system.

The first category of events is called Events and may include clinical events, for example medical interventions. Examples of clinical events may include, for example drug delivery, biopsy, surgery or physiotherapy. Clinical events and/or clusters of clinical events may be represented by markers placed within an Events band of the plot 44. In FIG. 3, markers 50a to 50e are arranged within the Events band.

The second category of events is called Labs and includes lab results. Lab results may include, for example, the results of blood or urine tests. Lab results and/or clusters of lab results may be represented by markers placed within a Labs band of the plot 44. In FIG. 3, markers 52a to 52h are arranged within the Labs band.

The third category of events is called Imaging and includes imaging results. Imaging results may include the results of imaging of any modality, for example computed tomography (CT), cone-beam CT, magnetic resonance (MR), position emission tomography (PET), single photon emission computed tomography (SPECT), X-ray or photography. Imaging results and/or clusters of image results may be represented by markers placed within an Imaging band of the plot 44. In FIG. 3, markers 54a to 54e are arranged within the Imaging band.

The fourth category of events is called Vitals and includes vital signs measurement results. Vital signs measurement results may include, for example, measurements of temperature, blood pressure, pulse rate and/or respiration rate. Vital signs results and/or clusters of vital signs results may be represented by markers placed within a Vitals band of the plot 44. In FIG. 3, markers 56a to 56e are arranged within the Vitals band.

For each of the Events, Labs, Imaging and Vitals categories, the use of the markers to represent events and/or clusters is described further below with reference to the display process of FIG. 2.

The horizontal time axis 46 and the vertical list of categories 48 may be together considered to form a view space. The markers 50a to 56e and 60a to 60d are arranged within the view space.

The timeline is marked in intervals that change across the length of the timeline. Vertical lines 47 are used to delineate days, weeks or years depending on the local scale. In the example of FIG. 3, the vertical lines 47 are solid lines.

A time marker 49 marks a current or selected time on the time axis. The time marker 49 is displayed as a vertical dotted line which extends for the full vertical axis. It may therefore be easy to relate the position of the markers to the current or selected time.

We now turn to the flow chart of FIG. 2 and consider the use of the method represented in FIG. 2 to create a medical display, for example the plot 44 of FIG. 3.

At stage 30 of FIG. 2, the acquisition circuitry 24 obtains information relating to a patient from data store 20. In other embodiments, the acquisition circuitry 24 may obtain information relating to any suitable human or animal subject.

The information comprises medical event data. The medical event data comprises a plurality of data points, each having at least one associated time value (for example, a time point or time range). Each data point may be considered to represent at least part of an event.

Each data point may comprise or relate to any item of medical information, for example a clinical note, a nursing note, a set of imaging data, a set of imaging measurements, a set of lab result data, a set of patient monitoring data, a set of vital sign data, a prescription, a medication record, data obtained from the patient, data obtained from a medical device, a summary report, a medical history report, a case conference report, a billing report, a radiology report, a set of patient events, medication data, administration or records data or any other suitable set of recorded information relating to the patient or other subject.

In the present embodiment, the medical event data obtained by the acquisition circuitry 24 includes all the medical event data available for that patient, for example all the medical event data in the patient's medical records. The medical event data comprises all of a patient's lifetime.

In other embodiments, the medical event data obtained by the acquisition circuitry 24 may comprise a subset of the medical event data available for that patient, for example medical event data pertaining to treatment in a certain hospital, by a certain doctor, in a certain department or relating to a certain specialty (for example, oncology department) or within a certain time range of the patient's lifetime.

Although in the present embodiment the medical event data is obtained from data store 20, in other embodiments the medical event data may be obtained from any suitable data store or data stores, for example from multiple servers on a network. The medical event data may be gathered from a healthcare informatics system or from a variety of healthcare informatics systems.

The medical event data may be formatted in any suitable electronic format, for example any known format for electronic medical record data. DICOM structured reports are one such possible format. In some embodiments, data pertaining to different medical events may have different data formats.

At stage 32, the clustering circuitry 27 processes the information obtained by the acquisition circuitry 25 at stage 30 to obtain a plurality of episodes. Episode detection is performed to find the start and end events of one or more time periods relating to the patient. Events corresponding to a start and end of a period of care may be matched to define an episode of care. For example, the clustering circuitry 27 may determine a hospital treatment episode by using a hospital admittance event and a hospital discharge event to determine a start and end of the episode. The clustering circuitry 27 may determine a treatment episode by using a treatment start event and a treatment event to determine a start and end of the episode.

Any suitable method of episode detection may be used. Episodes may represent any appropriate time periods, for example any time periods that are represented in the patient's medical record. The clustering circuitry 27 uses data included in the information obtained at stage 30 (for example, hospital admission and discharge records) to determine the episodes.

In other embodiments, the episodes are predefined within the information obtained at stage 30 and are imported by the clustering circuitry 27.

In some embodiments, there may be a hierarchical structure of episodes. An individual episode may be divided into sub-episodes. For example, an episode of hospitalization may contain sub-episodes that represent different phases of treatment.

At stage 34, the display circuitry 28 classifies each data point of the information obtained at stage 30 into at least one category. In the present embodiment, the display circuitry 28 classifies each data point into one of four categories: Events, Labs, Imaging or Vitals. In some circumstances, a given data point may be classified into more than one of the categories. In some embodiments, only a subset of the data points of the information are classified.

In other embodiments, the data points may be classified into any suitable categories. The data points may be classified by time. The data points may be classified by importance. The data points may be classified in dependence on one or more episodes, for example by classifying data points as inpatient or outpatient depending on whether the time values of the data points correspond to an episode of inpatient or outpatient treatment.

In some embodiments, the data points are classified by a frequency of data acquisition. For example, data acquisition may comprise at least one period of data acquisition at a higher data acquisition rate and at least one period of data acquisition at a lower data acquisition rate. Data points may be classified based on whether they were acquired in a period of higher data acquisition rate or a period of lower data acquisition rate.

The display circuitry 28 determines or receives values for a plurality of view parameters. The values for the view parameters are used by the display circuitry 28 to determine a non-linear view space onto which the classified data points are to be projected. In determining the view space, the display circuitry 38 also uses the categories into which the data points were classified.

The view parameters may be parameters of the view space, for example parameters relating to the non-linear timeline. The view parameters may include a time delta per pixel function, for example the time delta per pixel curve shown in FIG. 4b. The view parameters may comprise a time point of interest, which may also be described as a focal date, focal time, or focal date-time. The view parameters may comprise at least one zoom factor.

In the present embodiment, values for the view parameters are auto-generated. The projection circuitry 26 automatically selects a time point of interest based on relevancy calculations. For example, the time point of interest may be set as the current time or as the time of a last significant event. The projection circuitry 26 automatically selects the zoom factor to display the most recent episode of care for the patient.

In other embodiments, any suitable method of automatically generating values for the view parameters may be used. Values for time parameters, for example a range and focal time point of the view used in the view space, may be determined by the system to display the most relevant or recent information. In some embodiments, an auto-view function may be trained using deep learning, machine learning or artificial intelligence.

In some embodiments, different views may be presented in different settings or to different users. For example, a different view may be triggered based on a hospital admission or a transfer between wards.

In further embodiments, a user may set view parameters, for example to see detail of events at a particular time point. Values for at least some of the view parameters may be determined or adjusted by a user, for example a clinician.

In the present embodiment, the view space determined by the display circuitry 28 is the view space shown in FIG. 3. The view space comprises a horizontal time axis 46 which is configured to provide a non-linear representation of time, for example a time axis 46 in which time delta per pixel varies with x axis position as shown in FIG. 4b. The view space also comprises a vertical category axis 48 which is representative of the categories Episodes, Events, Labs, Imaging and Vitals. In other embodiments, the vertical axis may represent any suitable categories.

The projection circuitry 26 projects each data point onto the non-linear time axis 46. The projection circuitry 26 determining an x axis position for the data point using the time value associated with the data point and the function that defines the relationship between time delta per pixel and x axis position (for example, the function represented in FIG. 4b).

Since the time axis is non-linear, a given period of time may be represented by a different x axis distance depending where on the x axis it is positioned. For example, consider the non-linear axis of FIG. 3. The difference in time between −10 year and −1 year is represented by the same x axis distance as the difference in time between −1 year and −1 week.

By projecting the data points onto the time axis, time values are translated into x axis positions. In the present embodiment, all events are translated into the non-linear timeline. A single floating point number represents each event's position. In other embodiments, x axis positions may be expressed in any suitable manner using any suitable units of distance. For example, x axis positions may be expressed as pixel distances from the origin.

The projection circuitry 26 also projects each of the episodes determined at stage 32 into the view space. For each episode, x axis positions are determined for the at least the start time and the end time of the episode.

The clustering circuitry 27 performs a clustering procedure to cluster together certain of the data points into clusters of data points. The clustering procedure is performed using the x axis positions that were determined by projecting the data points onto the non-linear time axis. Where there are many events projected near to other events, these are combined into a cluster. Each cluster may represent a collection of events at different time points.

In the present embodiment, events are classified by category (Events, Labs, Imaging, Vitals) and the clustering circuitry 27 clusters events of the same category. Clustering is done on each category of events separately. The clustering circuitry 27 does not cluster events of different categories in a single cluster. In other embodiments, events in different categories may be clustered into a single cluster. In other embodiments, the events may be classified in any suitable manner and the clustering may be informed by the classification. For example, clustering may be performed on inpatient events separately from outpatient events. Clustering may be performed on data acquired at a higher data acquisition rate separately from data acquired at a lower data acquisition rate.

In the present embodiment, the clustering procedure comprises applying a 1D clustering algorithm to each category of data points. Any 1D clustering algorithm may be used to perform the clustering procedure, for example Jenks natural breaks optimization or kernel density estimation.

As a simple example of clustering, the clustering circuitry 27 may determine an x axis distance from each data point in a category to each other data point in that category. If data points fall within a threshold distance of each other, those data points may be clustered together to form a cluster.

In some circumstances, episodes are used to inform the clustering of other events. For example, it may be undesirable to cluster events across a boundary between episodes. The clustering procedure may prevent events falling within different episodes from being clustered together. In some embodiments, the clustering procedure may prevent events falling within different episodes from being clustered together unless the events themselves are clustered together.

The clustering procedure returns a set of clusters with each cluster containing a set of events. In some embodiments, it is possible for a cluster to contain a single event. In some embodiments, a cluster may contain nested clusters and events.

Because the clustering algorithm is based on x axis position rather than time, events that are further from the time of interest may be more likely to be clustered together than events that are nearer to the time of interest. For example, in regions of low granularity such as the regions at the far left end or right end of the plot of FIG. 3, events may be clustered together if they occur within a year of each other. In regions of high granularity such as the middle of the plot of FIG. 3, events may not be clustered together even if they occur within an hour of each other.

In FIG. 3, clusters of events are represented by rounded rectangular markers, and individual events are represented by diamond-shaped markers. A cluster is represented by a single marker. The length of the marker covers at least the projection of the first and last events in the cluster.

In regions of low granularity of clustering, a large number of data points may be clustered together as a single cluster and represented by a single marker. The number of markers used to represent the data may be low. At a very lowest level of granularity, all of the data points could be clustered together and represented by a single marker.

In regions of high granularity of clustering, each marker may represent a single data point or a small number of data points. The number of markers used to represent the data may be high. At a very highest level of granularity, no clustering may occur and each data point may be represented by a respective marker.

In FIG. 3, markers 50a, 50b, 50d and 50e are representative of clusters of clinical events. Marker 50c is representative of a single clinical event. Markers 52a, 52b, 52f, 52g and 52h are representative of clusters of lab result events. Markers 52c, 52d and 52e are representative of single lab result events. Markers 54a, 54b, 54c and 54e are representative of clusters of imaging events. Marker 54d is representative of a single imaging event. Markers 56a, 56b and 56e are representative of clusters of vital signs events. Markers 56c and 56d are representative of single vital signs events.

In some embodiments, certain events are classified as critical events. The clustering algorithm is configured such that it does not cluster a critical event together with any other event. The critical event is always presented as a single event no matter where it occurs on the time axis. For example, a critical event that is representative of a patient's heart attack may always be displayed on its own and not clustered with any other event.

Episodes may be considered to be special types of cluster. An episode typically has a set of child events that happen in between the start and the end of the episode. The child events may be events of any category. Episodes may be determined using events from any of all categories. The child events may be displayed as part of the cluster and/or may be displayed in a separate category.

In the embodiment of FIG. 3, episodes are plotted as a separate category that is shown in addition to the categories of Events, Labs, Imaging, Vitals. In other embodiments, episodes are plotted in the timeline within existing categories (for example, Events, Labs, Imaging, Vitals). In other embodiments (for example, the embodiment of FIG. 3), episodes are plotted as a separate category.

In some embodiments, multiple episodes may be combined into clusters of episodes. The clustering circuitry 27 is configured to cluster episodes using a clustering algorithm. The clustering algorithm used to cluster episodes may be the same clustering algorithm that is used to cluster events, or it may be a different clustering algorithm.

Clustering is performed so that when a group of events lie far from the current time focus point they do not overload the timeline and do not obscure adjacent events. Clustering and aggregation may keep the displayed elements relevant to a current context.

By performing clustering in non-linear view space, the clustering may be more granular (showing more detail) at times that are relatively near to the time point of interest, and may be less granular (showing less detail) at times that are further from the time point of interest. The change in granularity of clustering may be achieved without having to define different clustering algorithms or different clustering parameter values for different parts of the timeline. The granularity of clustering may vary smoothly along the timeline.

The use of episodes may provide contextual information to a user.

At stage 36, the clustering circuitry 27 summarizes the events in each cluster to generate a cluster summary. The cluster summary is a short piece of text. In the present embodiment, the cluster summary is displayed in a pop-up text box which appears when the user selects or hovers over the marker representing the cluster. The text box may be referred to as a tooltip.

The clustering circuitry 27 summarizes the events in each cluster using a cluster summary generation algorithm. The cluster summary generation algorithm is used to analyze all events for a cluster and generate a relevant summary text. The cluster summary generation algorithm may summaries the set of events that are clustered together.

The cluster summary generation algorithm may apply an importance weighting to each of the events in the cluster. In some embodiments, the summary is the description of the event with the highest importance. Important events may be flagged to ensure that information about the important events is propagated to a parent cluster. In some embodiments, less useful information is more aggressively clustered.

Display may be dependent on event saliency. In some embodiments, the cluster summary generation algorithm puts a higher weighting on events that are considered to be outliers than events that are considered to be routine. For example, the cluster summary generation algorithm may consider abnormal lab results to be more important than normal lab results.

In some circumstances, diagnosis results or treatment outcome may be prioritized in summary text, because diagnosis results or treatment outcome may be likely to be useful at a higher level.

In some embodiments, the cluster summary generation algorithm uses categorization strategies to summarize the cluster. Categorization strategies may include, for example, using importance weighting to ensure that the summary covers the most important event or events.

The cluster summary generation algorithm used may vary depending on the category of the events being summarized (for example, Events, Labs, Imaging or Vitals). Rules for summarizing may be category specific. For example, a summary of pharmaceutical events may be different from a summary of imaging events. Different drugs may have different importance values. A high importance value for a particular piece of information may increase the likelihood that the piece of information will be included in the summary.

In some embodiments, machine learning is used to generate smart summary text.

At stage 38, the display circuitry 28 uses the projected events and episodes from stage 34 and the summaries from stage 36 to generate a display. In the present embodiment, the display is the display 44 illustrated in FIG. 3. The display 44 is shown on display screen 16. In other embodiments, any suitable display may be generated. The display may be described as a smart timeline view.

The display circuitry 28 draws the non-linear timeline 46 along the x axis and the category list along the y axis. The display circuitry 28 draws markers 50a to 56e to represent events and clusters of events. Each marker is displayed in the band of the display 44 that is representative of its category. The display circuitry 28 draws markers 60a to 60d to represent episodes in the Episodes band of the display 44.

At stage 40, the display circuitry 28 receives a user input via a user interface, for example via a mouse or other input device 18. The user input may also be described as a user interface event.

In the present embodiment, the user input may be a pan command, a zoom command, a mouse hover over a cluster, a view command, or a double click on a selected cluster. In other embodiments, any suitable user input may be received.

At stage 42, the display circuitry 28 retrieves or calculates values for a set of view parameters in dependence on the user input. The view parameters may be the same as those used at stage 34, or may differ from those used at stage 34. The value for at least one of the view parameters may change in dependence on the user input.

We consider some specific examples of user inputs below. In other embodiments, any suitable user inputs may be received, and the values for the view parameters may be updated in any suitable manner.

When the values for the view parameters have been updated, the flow chart of FIG. 3 returns to stage 34. At stage 34, the projection circuitry 26 projects the events into the a new view space that has been determined in accordance with the updated view parameters.

The clustering circuitry 27 applies the clustering procedure to the new projection of the events. If the view space has changed, the clustering of the events may also change.

The flow chart proceeds to stage 36. At stage 36, the clustering circuitry 27 summarizes the events in each new cluster. At stage 38, the display circuitry 28 redraws the display 44 in accordance with the updated view parameters and the updated display is shown on display screen 16.

Stages 40, 42, 34, 36 and 38 may be repeated each time a user provides a user input via the user interface.

We now consider specific examples of user inputs that may be received at stage 40 via a mouse or other input device 18.

One example of a user input is a pan command. The pan command may be a command to move the timeline to the left or right, such that the timeline is centered on a different time.

In the present embodiment, the timeline is always centered on the time of interest, which may be also be described as a focal time position. When the display circuitry 28 receives the pan command, the display circuitry 28 sets a new focal time position in accordance with the pan command. For example, if the user has panned right along the timeline, the display circuitry 28 sets a later focal time position than that currently displayed.

We turn again to the plot of FIG. 4b, which shows time delta per pixel. When the pan command is received, it may be considered that the curve 72 of FIG. 4b and the focal point 74 of FIG. 4b stay the same with regard to the x axis. However, positions on the x axis of FIG. 4b map to different times. The time delta per pixel is at a minimum at the new focal time point.

When stage 34 is repeated for the new view parameters, the events are projected onto a new non-linear timeline which is now centered on the new focal time position. The change in focal time position may change which events are clustered and which are shown as individual events. The granularity of clustering at or near the new focal time point may be higher than was previously the case. The granularity of clustering at the previous focal time point may be lower than was previously the case.

In other embodiments, a further pan command may be provided which moves the timeline relative to the screen display without changing the time point of interest and/or without changing any values for time delta per pixel along the timeline.

We now consider the case in which the user provides a zoom command. When the user provides a zoom command, the focal time position does not change. In response to the zoom command, the display circuitry 28 decreases a time delta per pixel at the focal time position to increase the scale at the focal time position.

We considering again the plot of FIG. 4b. The lowest point 74 on the curve (which represents a time point of interest) may also be referred to as a central control point. Zooming moves the central control point 74, decreasing the time delta per pixel at the central control point.

In the present embodiment, when the lowest point 74 is made lower, the rest of the curve 72 is interpolated so that the end points stay the same as previously. A bias factor is set to change the shape of the curve which changes the distribution. In other embodiments, the whole curve 72 may be moved downwards in response to a zoom command.

The zoom command changes the view parameters of the non-linear timeline by changing the scale around the focus time position. The projection circuitry 26 projects the events into the view space using the new non-linear timeline. The clustering circuitry 27 applies the clustering procedure to the newly projected events. The granularity of clustering at or near the focal time position may be increased due to the change of scale.

Although a zoom in command (increase of scale) is described above, the zoom command may also be a zoom out command in which a time delta per pixel at the focal time point is decreased in response to the zoom command.

In some embodiments, the user gives an instruction to change a scale of a selected part of the time axis that does not relate to the time point of interest. The display circuitry 28 may maintain a scale used for the time point of interest while changing the scale of the selected part of the time axis. The display circuitry 28 may maintain a granularity of clustering at the time point of interest while changing a granularity of clustering at the selected part of the time axis.

When a pan or zoom command is given, the view shown by the display circuitry may typically smoothly animate between states. View parameters may be interpolated to give a smooth transition. For example, the focal time point or level of zoom may be changed smoothly over time. Parameters may be changed slowly enough that the user can see the change. By displaying a change of state as an animation, the change may be more easily understandable by the user.

As has already been described above, if the user input comprises hovering a mouse over a marker representing a cluster, the display circuitry 28 displays a tooltip which comprises a short text message giving summary information for that cluster. If the user input comprises hovering a mouse over a marker representing an event, the display circuitry 28 may display a tooltip comprising a short text message giving information for that event. In other embodiments, a tooltip may be displayed when a marker representing a cluster or event is selected, for example by a single click of a mouse or by any other suitable selection method.

In the present embodiment, when the mouse is not hovering over a cluster, then a vertical line marker 49 may be displayed over the timeline as shown in FIG. 3. In other embodiments, a vertical line marker or other time marker may be used in any suitable conditions. In some embodiments, more than one vertical line marker or other time marker may be placed. In some embodiments, a text box displays the date corresponding to the vertical line marker and/or the amount of time that has passed to the current date or to the focal time point.

We consider the case when the user input is a double click on a marker representing a cluster. In the present embodiment, if the display circuitry 28 receives a user input that is a double click on a cluster, the display circuitry 28 sets the focal time point and the time delta per pixel such the cluster is automatically expanded into its component events. For example, the display circuitry 28 may set the focal time point at a time that is midway between a start time of the cluster and an end point of the cluster. The display circuitry 28 may set a zoom level which is such that all of the events that were previously included in the cluster are now shown as individual events.

The display circuitry 28 may display the expansion of the cluster as a smooth animation between the non-expanded and expanded states. Displaying the expansion as an animation may help the user to understand the expansion process.

The display circuitry 28 may retain previous settings of view parameters (for example, focal point or zoom level) so that a user may revert back to the previous values.

The method described above with reference to FIG. 2 provides a non-linear timeline for displaying and summarizing patient events. It may give both focus and context to the visualization. A high-fidelity view of relevant events at a particular time point is provided, while other events are summarized.

Events in the patient's history that fall outside a priority time period may be displayed to the user without requiring user interactions to explore the patient's history. In contrast, some known methods display only a given time window and events outside the current time window are not displayed. Alternatively, in some known methods that show the whole of a patient's history and therefore provide adequate historical data, precision and context on events at a focal point may be lost.

In some circumstances, a user may be presented with relevant information without having to interact with the display. In some circumstances, a number of user interactions may be reduced. The user may be presented with clusters of less-important information. The user may have the ability to expand clusters if desired to obtain more detailed information.

In the embodiment described above with relation to FIGS. 2, 3 and 4b, there is a single time point of interest on the time axis. A region around the time point of interest is displayed at a large scale and is displayed in the center of the plot 44. Scale decreases with distance from the time point of interest.

In other embodiments, more than one time point may be of interest. For example, a clinician may want to view events at or near today's date at a high resolution, and may also want to view events at or near a patient's surgery at high resolution. The clinician may be less interested in events that occur between the date of the surgery and today's date.

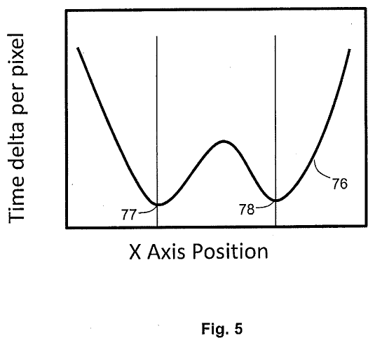

FIG. 5 shows a plot of time delta per pixel against x axis position for a scenario in which there are two time points of interest 77, 78. The lowest time delta per pixel (and therefore the largest scale) occurs both at point 77 and point 78. In between point 77 and point 78, the time delta per pixel increases. The time delta per pixel also increases for positions on the x axis that are below point 77 or above point 78.

A second focal point is used to display two periods of time with high fidelity while compressing the time between them. The system sets up the non-linear function to make the events around both focal time point clear.

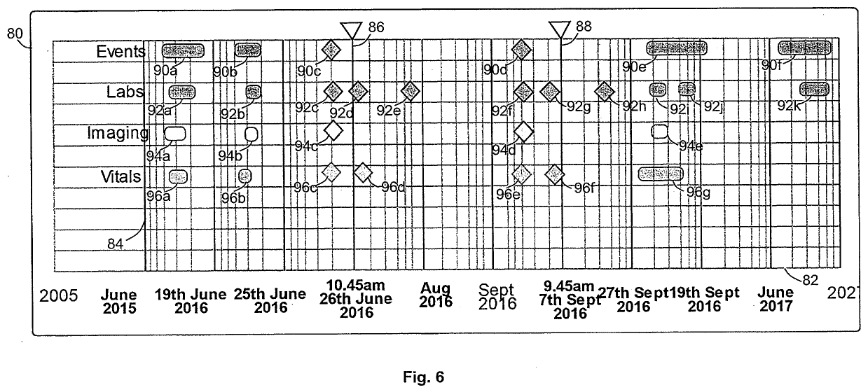

FIG. 6 shows an example of a plot 80 in which the non-linear time function is selected to show two focal time points at higher time resolution. A category axis 84 is plotted against a non-linear time axis 82. The two focal time points are indicated by time markers 86, 88. Time marker 86 is positioned at 10.45 am 26 Jun. 2016. Time marker 88 is positioned at 9.45 am 7 Sep. 2016.

An Events band of plot 80 includes markers 90a, 90b, 90e, 90f that are representative of clusters, and markers 90c, 90d that are representative of single events. A Labs band of plot 80 includes markers 92a, 92b, 92i, 92j, 92k that are representative of clusters, and markers 92c, 92d, 92e, 92f, 92g, 92h that are representative of single events. An Imaging band of plot 80 includes markers 94a, 94b, 94e that are representative of clusters, and markers 94c, 94d that are representative of single events. A Vitals band of plot 80 includes markers 96a, 96b, 96g that are representative of clusters, and markers 96c, 96d, 96e, 96f that are representative of single events.

In this example, the granularity of clustering is high near both of the time points of interest 86, 88, and so events near each of the time points are likely to be represented as individual events and not clustered. Events that are further from the time point are more likely to be clustered and to be represented as part of a cluster. Although not shown in FIG. 6, it may also be the case that events between the two time points of interest are more likely to be clustered than events at the time points themselves.

In other embodiments, there may be more than two time periods of interest. In some embodiments, a different time delta per pixel may be used for each of the time period of interest (a plot of time delta per pixel against x position may have two or more troughs of different depths).

By using a larger scale at two (or more) time periods of interest, a clinician may be able to tailor a view to see information about multiple periods, for example multiple important events or multiple periods of treatment. The clinician may be less interested in what has occurred between the periods of interest. The use of a non-linear timeline with more than one time period of interest may provide a flexible and adaptable display.

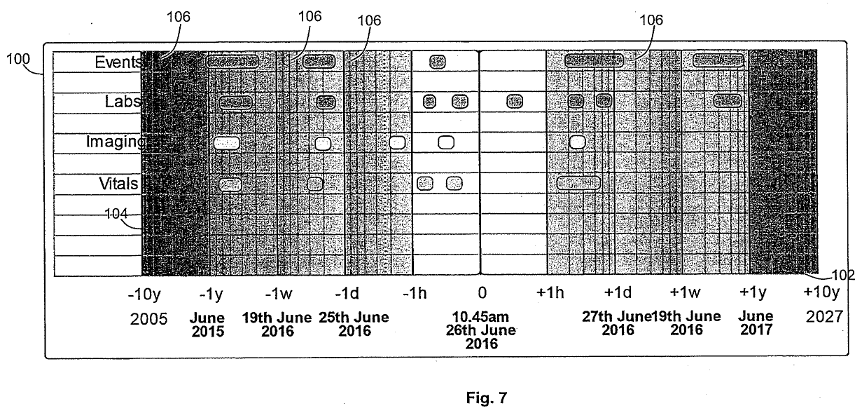

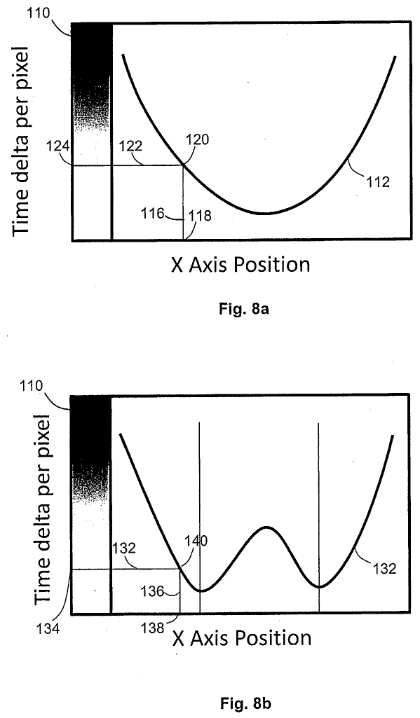

FIGS. 7, 8a and 8b relate to an embodiment in which a background color or texture of the view space is changed to give hints to the user with regard to the time density for different regions of the view space.

In the embodiments of FIG. 3 and FIG. 6, the scale of the timeline at each position on the timeline is indicated to the user by the time markings on the time axis 46, 82. However, it may be helpful for the user to have a further visual indication of the changes in time delta per pixel along the length of the time axis.

FIG. 7 shows a display 100 having a view space in which a background color intensity is changed to show a variation in time density. The view space comprises a non-linear time axis 102 and a category axis 104. A color of the view space (shown in FIG. 7 as greyscale) varies with position along the x axis. Where the scale of the time axis is large, a lighter background color is used. Where the scale of the time axis is smaller, a darker background color is used. In other embodiments, any type of visual effect (which may include, for example, color, texture or pattern) may be used to differentiate parts of the non-linear time axis having different scales.

In the embodiment of FIG. 7, a gradient table is used to define colors and time delta per pixel is used to index the table.

FIG. 8a is a schematic diagram representing the use of a gradient table that relates time delta per pixel to background color. FIG. 8a combines a gradient table 110 with a schematic plot of time delta per pixel against x axis position similar to that shown in FIG. 4a. A line 112 represents time delta per pixel as a function of x position. For each position on the x axis, the time delta per pixel may be determined from the function represented by line 112, and a color may then be selected from the gradient table based on the value for time delta per pixel. One example is shown in FIG. 8a. A line 116 connects an x axis position 118 to its corresponding position 120 on line 112. A line 122 then connects position 120 on line 112 to its value 124 for time delta per pixel on the vertical axis. Line 122 also cuts through the gradient table 110, indicating a color value from the gradient table 110.

FIG. 8b is a schematic diagram representing the use of a gradient table 130 with a schematic plot of time delta per pixel against x axis position. In FIG. 8b, a line 132 represents time delta per pixel as a function of x position. Line 132 has two minima that are representative of two time periods of interest. A line 136 connects an x axis position 138 to its corresponding position 140 on line 132. A line 142 then connects position 140 on line 132 to its value 144 for time delta per pixel on the vertical axis. Line 122 also cuts through the gradient table 130, indicating a color value from the gradient table 130.

In the embodiments shown in FIGS. 7, 8a and 8b, the table is used to select the color (for example, a color brightness and/or intensity). In other embodiments, colors may be alternated along the timeline. For examples, colors may be alternated by week or by year.

The use of background colors may give the user an immediate visual cue that they are viewing a non-linear timeline rather than a linear timeline. It may assist the user in navigating the non-linear timeline. The background color or texture may be chosen to make the non-linearity of the timeline clear.

Furthermore, vertical lines 106 are used to delineate days, weeks or years depending on the local scale.

Plots of time delta per pixel versus x axis position are illustrated in FIGS. 4a, 4b, 5, 8a and 8b for the purposes of illustration. We note that in the present embodiment the time delta per pixel curve is not displayed to the user. The time delta per pixel curve is a schematic drawing of an internal function that relates time delta per pixel to position on the x axis. The plots of FIGS. 4a, 4b, 5, 8a and 8b may provide a representation of the internal workings of the display circuitry 28.

The display circuitry 28 stores a function that relates time delta per pixel versus x axis position. Any suitable representation of the function may be used. The function is updated in response to user input. The user need not see the plot of time delta per pixel versus x axis position in order to provide an input that changes the function.

References to time points above may also refer to extended periods of time. For example, a reference to a time point of interest may refer to a period of time that is of interest. The period of time that is of interest may be of any appropriate length, for example an hour, a day, a week, a month or a year.

Certain embodiments provide a medical imaging apparatus comprising an interactive timeline view where the time axis is non-linear, where clusters of events are determined based upon their projection into the non-linear timeline, and where a collection of markers are plotted on the view that correspond to an event or collection of events at different time points.

Certain embodiments provide a medical imaging apparatus comprising a module that gathers events relating to a patient from a variety of healthcare informatics systems, where the module finds events corresponding to the start and end of a period of care, where these events are matched to define an episode of care, and where these episodes are used in an application to display contextual information to a user.

The timeline may correspond to a portion of a patient's lifetime and the events may be related to the interactions with a healthcare system. The user may be able to set view parameters to see the detail of the events at a particular time point. The range and focal time point of the view may be determined by the system to display the most relevant or recent information. Episodes may be displayed upon the timeline at a scale relevant to the position on the non-linear timeline. The background color or texture may be chosen to make the non-linearity of the timeline clear. The summary text for a cluster may be determined using importance weighting of the details of all the events that correspond to a cluster. There may exist multiple focal time points and the system may set up the non-linear function to make the events around both time points clear. The contents of the summary text may be displayed in a pop up box when the user selects or hovers over a marker.

Certain embodiments may provide a medical information system comprising an acquisition circuit configured to acquire an information concerning a patient from a memory; a display configured to display the information concerning the patient along a time axis; and a processing circuitry configured to classify the information concerning the patient, and change a granularity of the time axis on the based the classification the information concerning the patient.

The processing circuitry may be configured to classify the information concerning the patient as inpatient or outpatient.

The processing circuitry may be configured to set a base period to the time axis, provide an instruction of change a granularity of the time axis, and keep the granularity of either the base period or a range including the base period in case of change a granularity of the time axis of expect for the base period.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms; furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical information system comprising:
acquisition circuitry configured to acquire information concerning a subject from at least one memory, the information concerning the subject comprising a plurality of data points each having an associated time value;
display circuitry configured to display the information concerning the subject along a time axis; and
processing circuitry configured to:
classify the information concerning the subject; and
determine a granularity of clustering of the data points on the time axis in dependence on the classification of the information concerning the subject, such that data points in different regions of the time axis are clustered with different granularity.

2. A system according to claim 1, wherein the classifying of the information comprises classifying at least part of the information in accordance with its position on the time axis.

3. A system according to claim 1, wherein the processing circuitry is configured to classify at least part of the information as relating to inpatient treatment or outpatient treatment.

4. A system according to claim 1, wherein the processing circuitry is configured to classify at least part of the information by frequency of data acquisition.

5. A system according to claim 1, wherein the processing circuitry is configured to:
determine a time period of interest on the time axis;
provide an instruction to change a scale of at least part of the time axis; and
maintain a granularity of clustering and/or scale of the time period of interest while changing a scale of a further part of the time axis.

6. A system according to claim 1, wherein the time axis is non-linear such that different regions of the time axis are displayed at different scales.

7. A system according to claim 6, wherein the processing circuitry is configured to perform a clustering procedure on the time points based on the projection of the time points onto the non-linear time axis, such that time points in regions of smaller scale undergo greater clustering than time points in regions of larger scale.

8. A system according to claim 6, wherein a scale of the time axis varies continuously with distance along the time axis.

9. A system according to claim 1, wherein at least one time period of interest on the time axis is displayed at a larger scale than other regions of the time axis.

10. A system according to claim 1, wherein at least two time periods of interest on the time axis are displayed at a larger scale than other regions of the time axis, and a region of the time axis between the at least two time period of interests is displayed at a smaller scale than each of the time periods of interest.

11. A system according to claim 1, wherein the data points are representative of at least one of clinical events, laboratory results, pharmaceutical events, imaging events, patient events, clinical notes, nursing notes, patient monitoring data, vital sign data, prescriptions, medication records, patient data, medical device data, summary reports, medical history reports, case conference reports, billing reports, radiology reports, interactions with at least one healthcare system, and wherein the classifying of the information comprises classifying data points as at least one of clinical events, laboratory results, pharmaceutical events, imaging events, patient events, clinical notes, nursing notes, patient monitoring data, vital sign data, prescriptions, medication records, patient data, medical device data, summary reports, medical history reports, case conference reports, billing reports, radiology reports, interactions with at least one healthcare system.

12. A system according to claim 1, wherein the classifying comprises classifying data points by at least one of importance, saliency, whether an event or measurement represented by the data point is an outlier.

13. A system according to claim 1, wherein displaying the data points along the time axis comprises, for at least some of the data points, positioning markers that are representative of the data points on the time axis and/or positioning markers that are representative of clusters of data points on the time axis.

14. A system according to claim 13, wherein the time axis comprises at least one region of higher granularity of clustering and at least one region of lower granularity of clustering, such that each marker in the lower granularity region represents a greater number of data points than each marker in the higher granularity region.

15. A system according to claim 1, wherein the processing circuitry is configured to identify one or more episodes, each episode corresponding to a respective time period on the time axis, and, for the or each episode, to position a marker that is representative of the episode on the time axis.

16. A system according to claim 1, wherein the processing circuitry is configured to generate a respective summary text for each of a plurality of clusters of data points, wherein the clusters are represented by markers on the time axis.

17. A system according to claim 1, wherein the processing circuitry is further configured to receive at least one input signal from a user and to adjust the time axis and/or a granularity of clustering based on the at least one input signal.

18. A system according to claim 17, wherein adjusting the time axis comprises adjusting at least one of:
a portion of the time axis that is visible;
a scale of at least part of the time axis;
a rate of change of scale along the time axis;
a time period of interest.

19. A medical information system according to claim 1, wherein displaying the information along the time axis comprises applying a visual effect to distinguish regions of the time axis having different scales, optionally wherein the visual effect comprises a color and/or a texture.

20. A system according to claim 1, wherein the at least one memory comprises or forms part of one or more healthcare informatics systems.

21. A medical information display method comprising:
acquiring information concerning a subject from at least one memory, the information concerning the subject comprising a plurality of data points each having an associated time value;
displaying the information concerning the subject along a time axis; and
classifying the information concerning the subject; and
determining a granularity of clustering of the data points on the time axis in dependence on the classification of the information concerning the subject, such that data points in different regions of the time axis are clustered with different granularity.
